(19) United States
(12) Patent Application Publication
(10) Pub. No.: US 2016/0310324 A1
(43) Pub. Date: Oct. 27, 2016

(54) GOGGLE-TYPE PATIENT-CUSTOMIZED EYEBALL SHIELD APPARATUS FOR USE IN RADIATION THERAPY

(71) Applicant: THE CATHOLIC UNIVERSITY OF KOREA INDUSTRY-ACADEMIC COOPERATION FOUNDATION, Seoul (KR)

(72) Inventors: Joo Young Jung, Gyeonggi-do (KR); Do Kun Yoon, Gyeonggi-do (KR); Chang Uk Kim, Seoul (KR); Tae Suk Suh, Seoul (KR)

(21) Appl. No.: 14/417,756

(22) PCT Filed: Dec. 2, 2014
(86) PCT No.: PCT/KR2014/011676

§ 371 Date: Nov. 17, 2015

(30) Foreign Application Priority Data
Aug. 29, 2014 (KR) 10-2014-0114060

(51) Int. Cl. A61F 9/02

(52) U.S. Cl. CPC A61F 9/025

(57) ABSTRACT

The present invention provides a shield apparatus for protecting eyeball structures in radiation therapy for a tumor in an eye portion, particularly an eyelid(eyelid) or the conjunctiva. The shield apparatus is a goggle-type patient-customized eyeball shield apparatus for use in radiation therapy, including a pair of lens frames connected by a center connection part, temples, and nose pads. The goggle-type patient-customized eyeball shield apparatus for use in radiation therapy includes the lens frames configured such that open tops are formed by cutting away the upper portions thereof, respectively, and lens grooves are formed along the inner circumferences thereof, respectively; lenses inserted into the respective lens grooves through the open tops; and shields each fastened onto the surface of each of the lenses via any one of a plurality of fastening holes passing through the lens, and configured to shield radiation to be applied to eyeballs. An advantage arises in that the vertical relationship between a radiation beam and a treated portion can be changed or maintained because the bending angles of the lens frames and temples of the shield apparatus and the locations of the shields can be immediately controlled in accordance with the shape of the face of a patient and the locations of the eyes and pupils of the patient.

### TECHNICAL FIELD

The present invention relates to a shield apparatus for protecting eyeball structures in radiation therapy for a tumor in an eye portion, particularly an eyelid(eyelid) or the conjunctiva, and more particularly to a goggle-type patient-customized eyeball shield apparatus for use in radiation therapy, which is configured to be used in such a way as to be worn on a face like goggles, which enables the bending angles of temples and both lens frames, into which lenses are inserted, forming the shield apparatus, to be controlled, and which enables the locations of shields fastened onto the lenses to be freely changed.

### BACKGROUND ART

90% or more of tumors occurring near eyeballs, particularly in eyelids, are basal cell carcinomas (basal cell carcinomas), and include squamous cell carcinomas (squamous cell carcinomas), melanomas (melanomas), sebaceous carcinomas, meibomian gland carcinomas (sebaceous carcinomas, meibomian gland carcinomas), merkel cell carcinomas (merkel cell carcinomas), etc. Furthermore, tumors occurring near eyelids or in the conjunctivae are treated chiefly using external beam radiation therapy (external beam radiation therapy, EBRT), and the safety and effectiveness of the EBRT have been proved.

In the above-described EBRT(EBRT) for treating a tumor near an eyelid, an electron beam(electron beam) is used. Normal tissue, other than a tumor, near an eyelid is protected by an external shield. A conventional shield uses a contact lens-type shield, as disclosed in U.S. Pat. No. 5,487,394A entitled “Tungsten Eyeball Shields for Electron Beam Treatment.” This contact lens-type shield is made of tungsten and aluminum materials, has a hemispherical shape having a thickness of about 3.0 mm, and has been commercialized as a technology for shielding an optical structure. This is an embodiment of therapy that is preferable to the case where a lead shield having a thickness of 4.5 mm is used when therapy is performed using a radiation energy of 15 MeV.

However, a patient must directly attach and detach the contact lens-type shield to and from his or her eyeball. In this case, the following various problems occur.

First, in the case of the therapy using an electron beam, an unnecessary amount of radiation, as in backward scattering, is accumulated through interaction with the tungsten and aluminum materials and then transferred to normal tissue. Transmissivity and backward scattering related to this problem can be corrected by fabricating several shield models having different tungsten and aluminum thicknesses. However, it is difficult to make a therapy plan for the unnecessary amount of radiation for an eyeball disposed at a bottom portion below an eyelid, and the amount of radiation prescribed for an eyelid is also increased due to the shield.

Second, therapy using the EBRT(EBRT) is commonly performed about 20 times in a divided manner. The eyeball shield should be attached and detached each time. This method is problematic in that contagion management needs to be carefully performed in terms of hygiene.

Third, the contact lens-type shield directly inconveniences a patient. While radiation therapy is being performed, the gaze of a patient should be fixed on a one side location. However, when the shield is placed on an eye, the unnecessary movement of an eyeball occurs, thereby reducing the efficiency of therapy.

### DISCLOSURE

### Technical Problem

Accordingly, the present invention has been made keeping in mind the above-described problems, and an object of the present invention is to provide a goggle-type patient-customized eyeball shield apparatus for use in radiation therapy, which can be used in such a way as to be worn on a face in order to protect eyeball structures when external radiation therapy is performed on an eyelid (eyelid) or the conjunctiva, which can be worn in accordance with the shapes of the head and face of a patient by controlling the bending angles of lens frames and temples that form the shield apparatus, and which enables the locations of shields fastened onto lenses inserted into the lens frames to be changed so that the shields can be located above the centers of the eyeballs of the patient.

### Technical Solution

In order to accomplish the above objects, there is provided a goggle-type patient-customized eyeball shield apparatus for use in radiation therapy, the apparatus including a pair of lens frames connected by a center connection part, temples, and nose pads, the apparatus including the lens frames configured such that open tops are formed by cutting away the upper portions thereof, respectively, and lens grooves are formed along the inner circumferences thereof, respectively; lenses inserted into the respective lens grooves through the open tops; and shields each fastened onto the surface of each of the lenses via any one of a plurality of fastening holes passing through the lens, and configured to shield radiation to be applied to eyeballs.

The inward bending angles of both the lens frames may be controlled by the center connection part.

The connection part may include a central shaft disposed between both the lens frames, and configured to have sleeve fastening recesses formed in respective upper and lower portions of an outer circumference thereof; an upper rotating sleeve coupled to one of the lens frames, and placed around the upper portion of the central shaft; a lower rotating sleeve coupled to the other one of the lens frames, and placed around the lower portion of the central shaft; and fastening bolts inserted into the respective sleeve fastening recesses of the central shaft from the outsides of the upper and lower rotating sleeves and then fastened therein.

The patient-customized eyeball shield apparatus may further include lens fastening bolts coupled to respective upper outside portions of the lens frames and configured to fasten the lenses.

The temples may be connected to the respective lens frames in an artificial joint form so that outward bending angles can be controlled, and the artificial joint connection between the temples and the lens frames is performed via joint balls coupled to the respective upper outside portions of the lens frames and joint recesses formed in ends of the temples and configured to receive the joint balls.

### Advantageous Effects

In accordance with the goggle-type patient-customized eyeball shield apparatus for use in radiation therapy according to the present invention, first, advantages arise in that when an eyelid or the conjunctiva is treated, radiation shield management can be more rapidly and accurately performed, and contagion management can be easily performed.

Second, a secondary problem, such as an eyeball erythema, attributable to an increase in the amount of radiation transferred, as in backward scattering, can be eliminated. In this case, advantages arise in that radiation therapy can be activated in the field of eyeball treatment that is currently depending on surgical operation therapy and almost institutions that treat eyeballs using radiation may purchase and fabricate the shield apparatus.

Third, when the radiation goggle-type shield apparatus according to the present invention is used, advantages arise in that the economic burden of patients can be reduced and also massive profits can be created by hospitals and companies using the radiation shield apparatus, with the result that economic efficiency is increased.

Fourth, advantages arise in that the radiation shield apparatus can be easily commercialized and in that usability can be increased because almost institutions that treat eyeballs using radiation can easily use the radiation shield apparatus if companies that perform customized fabrication take a goggle-type apparatus into consideration.

Moreover, an advantage arises in that the vertical relationship between a radiation beam and a treated portion can be changed or maintained because the bending angles of the lens frames and temples of the shield apparatus and the locations of the shields can be immediately controlled in accordance with the shape of the face of a patient and the locations of the eyes and pupils of the patient.

### DESCRIPTION OF DRAWINGS

FIG. 1 is a perspective view of a goggle-type patient-customized eyeball shield apparatus for use in radiation therapy according to the present invention and an enlarged view of a principal part of the patient-customized eyeball shield apparatus;

FIG. 2 is a partial perspective view of the lens frame and connection part of FIG. 1 and an exploded perspective view of a shield; and

FIG. 3 is a longitudinal sectional view of the connection part taken along line A-A of FIG. 2.

### BEST MODE

Preferred embodiments of a goggle-type patient-customized eyeball shield apparatus for use in radiation therapy according to the present invention will be described in detail below with reference to the accompanying drawings. The present invention is not limited to the disclosed embodiments, but may be implemented in various forms. These embodiments are provided merely to make the disclosure of the present invention complete and to allow those having ordinary knowledge to completely understand the scope of the present invention.

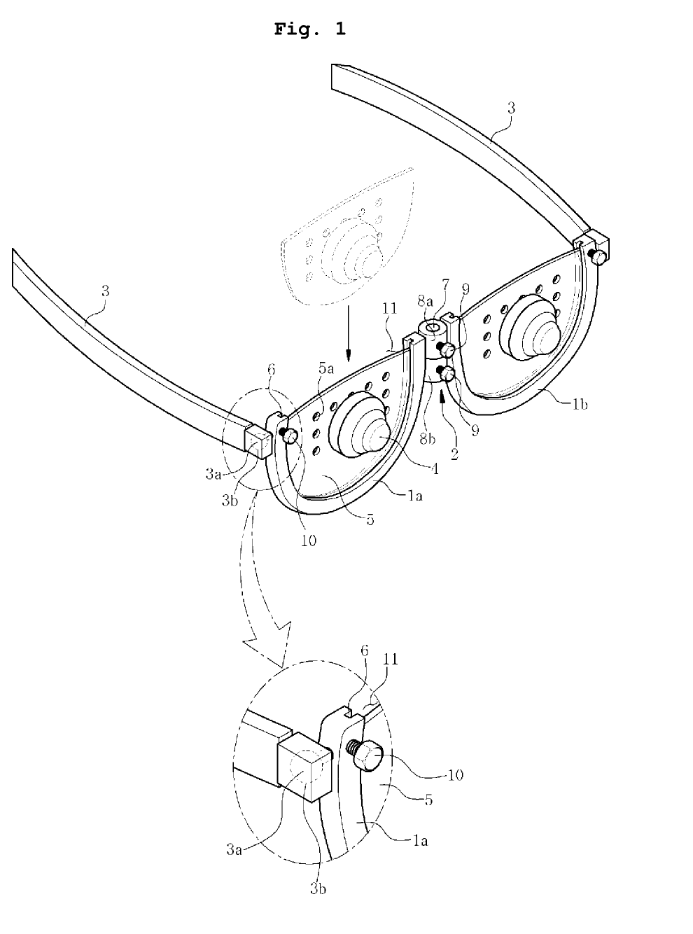

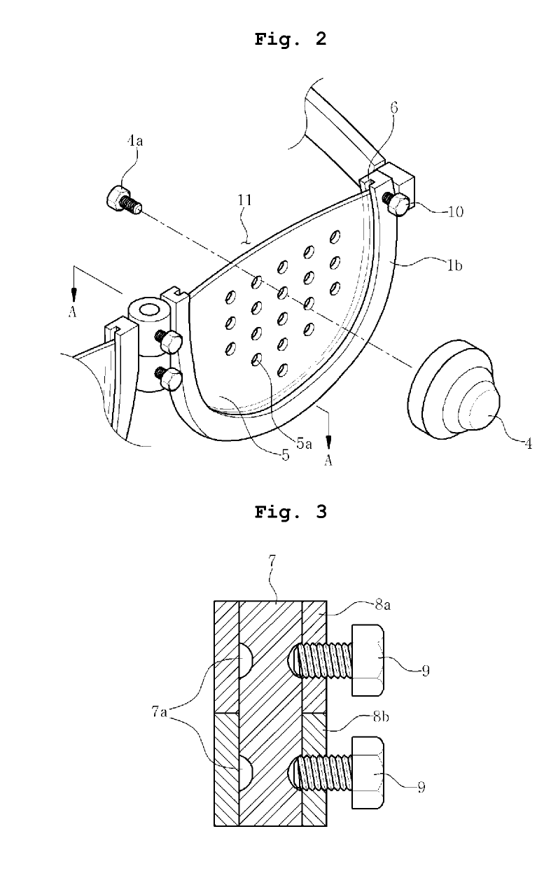

FIG. 1 is a perspective view of a goggle-type patient-customized eyeball shield apparatus for use in radiation therapy according to the present invention and an enlarged view of a principal part of the patient-customized eyeball shield apparatus, FIG. 2 is a partial perspective view of the lens frame and connection part of FIG. 1 and an exploded perspective view of a shield, and FIG. 3 is a longitudinal sectional view of the connection part taken along line A-A of FIG. 2.

As illustrated in FIGS. 1 to 3, the goggle-type patient-customized eyeball shield apparatus for use in radiation therapy according to the present invention includes a pair of lens frames (1a) (1b) configured to have respective nose pads formed thereon; a connection part (2) configured to connect the lens frames (1a) (1b) to each other; temples (3) connected to the outsides of the lens frames (1a) (1b), respectively; and lenses (5) installed in the respective lens frames (1a) (1b), and configured to have shields (4) fastened thereon. Although not illustrated, the nose pads may be installed on the lens frames (1a) (1b) or on the connection part (2) as desired, like those of typical glasses. The top portions of the lens frames (1a) (1b) are cut away, thereby forming respective open tops (11).

Lens grooves (6) into which the respective edges of the lenses (5) are inserted are formed along the inner circumferences of the lens frames (1a) (1b).

The connection part (2) includes a central shaft (7), a pair of upper and lower rotating sleeves (8a) (8b) and shield fastening bolts (9) so that bending angles by which the lens frames (1a) and (1b) are selectively bent inward and outward can be controlled. In this connection part (2), the upper rotating sleeve (8a) is placed around the upper portion of the central shaft (7) in which sleeve fastening recesses (7a) have been formed in the upper and lower portions of the outer circumference of the central shaft (7), and is coupled to one of the lens frames (1a). In order to allow the lens frames (1a) (1b) to be freely bent inward, the lower rotating sleeve (8b) is placed around the lower portion of the central shaft (7), and is coupled to the other lens frame (1b). The shield fastening bolts (9) that are passed and fastened through the outer circumferences of the upper and lower rotating sleeves (8a) (8b) are configured to be inserted up to the sleeve fastening recesses (7a) of the central shaft (7). Accordingly, inward bending angles are controlled in accordance with the angles of the facial portions of a patient near eyeballs by rotating the lens frames (1a) (1b) via the connection part (2), and are then fastened using the shield fastening bolts (9).

The temples (3) are connected to the lens frames (1a) (1b) in an artificial joint form. That is, joint balls (3a) are coupled to the upper outer portions of the respective lens frames (1a) (1b) so that the joint balls protrude outward. Joint recesses(3b) into which the joint balls (3a) are inserted are formed in the ends of the temples (3). Accordingly, the goggle-type shield apparatus can be conveniently worn in accordance with the size of the head of a patient because the bending angles by which the temples (3) are freely bent outward can be controlled by joint movement.

The lenses (5) are made of soft glass like spectacle lenses. The shields (4) are attached and fastened to approximately the central parts of the lenses (5). A plurality of fastening holes (5a) is formed throughout each of the lenses (5) at specific intervals, so that the lens (5) is fastened by a shield fastening bolt (4a) inserted into the lens through the fastening hole from the back after the lens (5) has been disposed above any one of the plurality of fastening holes (5a). Accordingly, the shields (4) can be accurately disposed in alignment with the pupils of the eyeballs of a patient having different locations by fastening the lenses (5) through appropriate fastening holes because the distance between the eyebrows and the locations of the pupils of the eyes vary depending on the patient.

After the lenses (5) have been inserted into the lens frames through the lens grooves (6), the locations of the lenses are controlled in accordance with a patient by slightly moving the lenses. Thereafter, it is preferred that the lenses (5) are securely fastened by respective lens fastening bolts (10) inserted into the outer upper portions of the lens frames (1a) (1b) so that they are not separated from the lens frames (1a) (1b).

### MODE FOR INVENTION

The shield apparatus of the present invention is configured such that the lenses (5), to which compensators (compensators), i.e., the shields (4), which are fabricated depending on the amount of energy used to treat a patient and the number of treatments have been attached on the surfaces of the lenses 5, are inserted into the goggle-type lens frames 1a and 1b. Accordingly, since the shapes of the shields vary depending on the shape of tumor tissue and the energy with respect to each patient, eyeballs are shielded using the shield apparatus in which the shields have been disposed on the lenses at variable locations according to the present invention when radiation therapy is performed.

Accordingly, the goggle-type patient-customized eyeball shield apparatus of the present invention for use in radiation therapy can handle the management of eyeballs and related normal tissue within a common energy range of 6 MeV or more without requiring a specific procedure.

Therefore, a secondary problem, such as eyeball erythema, attributable to an increase in the amount of radiation transferred, as in backward scattering, can be eliminated. In this case, radiation therapy can be activated in the field of eyeball treatment that is currently depending on surgical operation therapy.

Meanwhile, when the radiation shield apparatus is used as described above, the economic burden of patients can be reduced and also massive profits can be created by hospitals and companies using the radiation shield apparatus, with the result that economic efficiency is increased.

Furthermore, the radiation shield apparatus can be easily commercialized because it is fabricated in a goggle form. Furthermore, usability can be increased because almost institutions that treat eyeballs using radiation can easily use the radiation shield apparatus when companies that perform customized fabrication take mounting into consideration.

As described above, although the goggle-type patient-customized eyeball shield apparatus for use in radiation therapy according to the present invention has been described with reference to the illustrative drawings, it will be apparent that the present invention is not limited to the embodiments disclosed in the present specification and the drawings and various modifications and variations can be made by those skilled in the art without departing from the scope of the present invention.

### INDUSTRIAL APPLICABILITY

The patient-customized eyeball shield apparatus according to the present invention can be advantageously used as an eyeball shield apparatus that when an eyelid or the conjunctiva is treated, can more rapidly and accurately perform radiation shield management and also can easily perform contagion management.

Furthermore, the patient-customized eyeball shield apparatus according to the present invention can eliminate a secondary problem, such as an eyeball erythema, attributable to an increase in the amount of radiation transferred, as in backward scattering.

In this case, the patient-customized eyeball shield apparatus can be advantageously used as an eyeball shield apparatus that can activate radiation therapy in the field of eyeball treatment that is currently depending on surgical operation therapy and that may be purchased and fabricated by almost institutions that treat eyeballs using radiation.

Furthermore, the patient-customized eyeball shield apparatus according to the present invention can be advantageously used as an eyeball shield apparatus that can increase economic efficiency because the economic burden of patients can be reduced and also massive profits can be created by hospitals and companies using the radiation shield apparatus.

Furthermore, the patient-customized eyeball shield apparatus according to the present invention can be advantageously used as an eyeball shield apparatus that can be easily commercialized and that can increase usability because almost institutions that treat eyeballs using radiation can easily use the radiation shield apparatus if companies that perform customized fabrication take a goggle-type apparatus into consideration.

Moreover, the patient-customized eyeball shield apparatus according to the present invention can be advantageously used as an eyeball shield apparatus that can change or maintain the vertical relationship between a radiation beam and a treated portion can be changed or maintained because the bending angles of the lens frames and temples of the shield apparatus and the locations of the shields can be immediately controlled in accordance with the shape of the face of a patient and the locations of the eyes and pupils of the patient.

1. A goggle-type patient-customized eyeball shield
apparatus for use in radiation therapy, the apparatus comprising a pair of lens frames connected by a center connection part, temples, and nose pads, the apparatus comprising:
the lens frames configured such that open tops are formed by cutting away upper portions thereof, respectively, and lens grooves are formed along inner circumferences thereof, respectively;
lenses inserted into the respective lens grooves through the open tops; and
shields each fastened onto a surface of each of the lenses via any one of a plurality of fastening holes passing through the lens, and configured to shield radiation to be applied to eyeballs.

2. The patient-customized eyeball shield apparatus of claim 1, wherein inward bending angles of both the lens frames can be controlled by the center connection part.

3. The patient-customized eyeball shield apparatus of claim 2, wherein the connection part comprises:
a central shaft disposed between both the lens frames, and configured to have sleeve fastening recesses formed in respective upper and lower portions of an outer circumference thereof;
an upper rotating sleeve coupled to one of the lens frames, and placed around the upper portion of the central shaft;
a lower rotating sleeve coupled to a remaining one of the lens frames, and placed around the lower portion of the central shaft; and
fastening bolts inserted into the respective sleeve fastening recesses of the central shaft from outsides of the upper and lower rotating sleeves and then fastened therein.

4. The patient-customized eyeball shield apparatus of any one of claims 1 to 3, further comprising lens fastening bolts coupled to respective upper outside portions of the lens frames, and configured to fasten the lenses.

5. The patient-customized eyeball shield apparatus of any one of claims 1 to 3, wherein the temples are connected to the respective lens frames in an artificial joint form so that outward bending angles can be controlled.

6. The patient-customized eyeball shield apparatus of claim 5, wherein the artificial joint connection between the temples and the lens frames is performed via joint balls coupled to the respective upper outside portions of the lens frames and joint recesses formed in ends of the temples and configured to receive the joint balls.
